(19) United States
(12) Patent Application Publication
(10) Pub. No.: US 2017/0065660 A1
(43) Pub. Date: Mar. 9, 2017

(54) NUTRACEUTICAL COMPOSITION AND METHOD OF USE THEREOF

(71) Applicants: Linda Hale, Leesburg, VA (US); Joseph Bryan Crosswhite, Washington, DC (US)

(72) Inventors: Linda Hale, Leesburg, VA (US); Joseph Bryan Crosswhite, Washington, DC (US)

(21) Appl. No.: 14/845,962

(22) Filed: Sep. 4, 2015

(51) Int. Cl. A61K 36/9068; A61K 36/889; A61K 36/886; A61K 36/55; A61K 36/185; A61K 36/54; A61K 36/9066; A61K 36/752; A61K 36/898; A61K 36/73; A61K 36/88; A61K 33/00; A61K 31/07; A61K 31/375; A61K 31/592; A61K 31/593; A61K 31/355; A61K 31/122; A61K 31/51; A61K 31/525; A61K 31/455; A61K 31/675; A61K 31/519; A61K 31/714; A61K 31/4188; A61K 31/197; A61K 33/06; A61K 33/26; A61K 33/18; A61K 33/30; A61K 33/04; A61K 33/34; A61K 33/32; A61K 33/24; A61K 31/7004; A61K 31/14; A61K 36/63

(52) U.S. Cl. CPC A61K 36/9068; A61K 36/63; A61K 36/889; A61K 36/886; A61K 36/55; A61K 36/185; A61K 36/54; A61K 36/9066; A61K 36/752; A61K 36/898; A61K 36/73; A61K 36/88; A61K 33/00; A61K 31/07; A61K 31/375; A61K 31/592; A61K 31/593; A61K 31/355; A61K 31/122; A61K 31/51; A61K 31/525; A61K 31/455; A61K 31/675; A61K 31/519; A61K 31/714; A61K 31/4188; A61K 31/197; A61K 33/06; A61K 33/26; A61K 33/18; A61K 33/30; A61K 33/04; A61K 33/34; A61K 33/32; A61K 33/24; A61K 31/7004; A61K 31/14; A23L 1/3006; A23L 1/302; A23L 1/3002; A23V 2002/00

(57) ABSTRACT

A nutraceutical composition comprising water having an alkaline pH and a plurality of members of the group consisting of orange extract, vanilla extract, apple honey, agave syrup, apple cider vinegar, olive oil, coconut oil, liquid multivitamin, aloe vera juice, flax seed oil, hemp oil, and elephant garlic, wherein each of the plurality of members is present in an amount effective to ameliorate the effects of inflammation and raise energy levels when administered to a mammal.

### BACKGROUND OF INVENTION

Nutraceuticals, also known as phytochemicals, are natural, bioactive chemical compounds that provide numerous physiological benefits, including, inter alia, disease prevention and health promotion. Nutraceuticals are used to achieve both long-term and short-term health objectives. There are a huge, varied and almost limitless variety of nutraceuticals. Nutraceuticals contain at least one of the following dietary ingredients: a vitamin, a mineral, an herb or other botanical, an amino acid, a metabolite, constituent, extract, or combination of these ingredients. Common examples of nutraceuticals include beta-carotene (used to prevent cardiac arrest), ephedra (used to aid in weight loss), ginko biloba (used as a circulatory stimulant), goldenseal (used in the prevention and/or treatment of infectious diseases), valerian (used as a sedative and anxiolytic), ginseng (used for stress relief and restoration of homeostasis), and echinacea (used for prophylaxis and treatment of viral, bacterial and fungal infections).

They play a role in a plethora of biological processes, including oxidative stress prevention, cell proliferation, gene expression, safeguarding of mitochondrial integrity, prevention of chronic and life threatening diseases such as, for example, inflammation, diabetes, renal and gastrointestinal disorders, Alzheimer's disease, cardiovascular diseases, cancer, eye conditions, Parkinson's diseases and obesity, as well as numerous infections, postponement of the aging process (thus increasing life expectancy), amelioration of allergy symptoms, stabilization of immune system, thus, nutraceuticals are a low cost nutritional supplement, which the consumer can ostensibly use to manage their own health care, increasing or decreasing the use (or frequency of use) thereof as the consumer sees fit, based either on their own perceived needs or on the advice of a medical doctor.

A wide variety of nutraceuticals have previously been implemented in the treatment of various physiological and pathological conditions. For example, Konishi, U.S. Pat. No. 6,932,990 discloses a carbohydrate absorption inhibitor derived from the nutraceudical evening primrose seed. McPeak, U.S. Pat. No. 6,902,739 discloses formulations for treating an inflammatory disease or reducing an inflammatory reaction comprising via a fortified formulation comprising rice bran derivative and a fortification agent. Shashoua, U.S. Pat. No. 6,890,896 discloses a composition using various nutraceuticals to counteract harmful oxidative effects of reactive oxygen species and other free radicals. Giori, U.S. Pat. No. 6,881,426 discloses an extract of the nutraceutical echinacea to strengthen immune defenses of the lymphatic system. Mousa, U.S. Pat. No. 6,866,864 discloses a garlic-derived compound for the prevention and treatment of vascular-related disorders. Hong, U.S. Pat. No. 6,827,950 discloses a composition comprising extracts of the nutraceutical aralia to prevent cataracts, delay the development of cataract, and treat cataract. Nair, U.S. Pat. No. 6,818,324 discloses an extract of the nutraceutical anthocyanin for pain relief and anti-inflammation. Holub, U.S. Pat. No. 6,784,159 discloses the use of the nutraceutical soyasaponin B. sub. b for the treatment of polycystic kidney disease. Butters, U.S. Pat. No. 6,761,913 discloses an extract of celery seed for treatment and prevention of acute and chronic pain, inflammation and gastrointestinal irritation. Heeg, U.S. Pat. No. 6,733,798 discloses cranberry seed oil for the treatment of burned tissue. Garrity, U.S. Pat. No. 6,703,333 discloses a composition including the nutraceutical mangosteen for improving general health and wellness. Nair, U.S. Pat. No. 6,623,743 describes a nutraceutical composition comprising anthocyanins, bioflavonoids, phenolics or mixtures thereof from cherries for inhibiting oxidation. Cartwright, U.S. Pat. No. 6,585,998 discloses a nutraceutical composition including a tripeptoid component, a flavonoid component, guanidine hydrochloride, α-lipoic acid, an amino acid component, a brazilin component, catalase, and, optionally, vitamin E and selenium to maintain normal blood sugar levels and normal levels of non-enzymatic protein glycosylation. Henderson, U.S. Pat. No. 6,492,349 discloses a composition including the nutraceuticals glucosamine and chondroitin sulfate for the treatment and repair of connective tissue.

It is an object of the invention to provide a nutraceutical composition for the relief of physical symptoms and low energy levels caused by inflammation.

### SUMMARY OF INVENTION

The above and other objects are realized by the present invention, one embodiment of which relates to a nutraceutical composition comprising water having an alkaline ionized pH and a plurality of elderberry syrup, vanilla extract, orange extract, apple honey, agave syrup, apple cider vinegar, olive oil, coconut oil, liquid multivitamin, aloe vera juice, flax seed oil, hemp oil, and elephant garlic, wherein each of said plurality of ingredients is present in amount effective to ameliorate the effects of inflammation and raise energy levels when administered to a mammal.

Another embodiment of the invention concerns a method for the amelioration of the effects of inflammation and raising energy levels in a mammal comprising the administration thereto of the above described nutraceutical composition.

### DETAILED DESCRIPTION OF INVENTION

While the invention will be described and disclosed here in connection with certain preferred embodiments, the description is not intended to limit the invention to the specific embodiments shown and described here, but rather the invention is intended to cover all alternative embodiments and modifications that fall within the spirit and scope of the invention as defined by the claims included herein as well as any equivalents of the disclosed and claimed invention.

The present invention is predicated on the discovery that a plurality of elderberry syrup, vanilla extract, orange extract, apple honey, agave syrup, apple cider vinegar, olive oil, coconut oil, liquid multivitamin, aloe vera juice, flax seed oil, hemp oil, and garlic function together synergistically to ameliorate the symptoms associated with inflammation and raise energy levels to a greater extent than would be expected based on present day knowledge of the properties thereof, provided they are administered to a mammal in need thereof in a medium comprising water having an alkaline ionized pH; preferably in the range:

The preferred compositions are those containing all of the above components in the following proportions:
60-120 ounces water,
0.5-1 ounce orange extract,
0.5-1 ounce vanilla extract,
0.5-1 ounce elderberry syrup,
0.5-1 ounce apple honey,
1-2 ounces agave syrup,
0.5-1 ounce apple cider vinegar,
1.5-3 ounces olive oil,
1.5-3 ounces coconut oil,
1.5-3 ounces liquid multivitamin,
1.5-3 ounces aloe vera juice,
0.5-1 ounce flax seed oil,
0.5-1 ounce hemp oil,
1.5-3 ounces elephant garlic,
1.5-3 ounces cinnamon,
1.5-3 ounce turmeric,
1.5 3 ounces ginger, and
3 lemons

It is to be understood, however, that although optimum benefits are achieved when employing the above-described composition, significantly advantageous results are also achieved when employing less than all of the above ingredients.

For example, it has been discovered that compositions containing the following plurality of components in the stated proportions will yield excellent benefits:
60-120 ounces water
1.5-3 ounces olive oil
1.5-3 ounces coconut oil
1.5-3 ounces liquid multivitamin
1.5-3 ounces aloe vera juice
0.5-1 ounce flax seed oil
0.5-1 ounce hemp oil, and
1.5-3 ounces garlic.
1.5-3 ounces cinnamon
1.5-3 ounce tumeric
1.5 3 ounces ginger
3 lemons

Coconut oil contains so-called Medium Chain Triglycerides (MCTs)—which are fatty acids of a medium length. Most of the fatty acids in the diet are long-chain fatty acids, but the medium-chain fatty acids in coconut oil are metabolized differently. They go straight to the liver from the digestive tract, where they are used as a quick energy source or metabolized into ketone bodies, which have therapeutic effects on brain disorders e.g., epilepsy and Alzheimer's. The medium-chain triglycerides (MCTs) in coconut oil increase energy expenditure compared to the same amount of calories from longer chain fats. One study found that 15-30 grams of MCTs per day increased 24 hour energy expenditure by 5%. Almost 50% of the fatty acid content in coconut oil is lauric acid. When coconut oil is enzymatically digested, it also forms the monoglyceride, monolaurin. Both lauric acid and monolaurin kill harmful pathogens such as bacteria (e.g., Staphylococcus Aureus), viruses and fungi (e.g., Candida Albicans. The MCTs in coconut oil also increase the blood concentration of ketone bodies, which help reduce seizures in epileptic children, supply energy for the brain cells of Alzheimer's patients, thereby relieving symptoms thereof.

Olive oil: A study focused on the biological and clinical effects of olive oil, published in the journal Pharmacological Research found that people who regularly consume olive oil are much less likely to develop cardiovascular diseases, including hypertension, stroke, and hyperlipidemia. The same study also found that regular olive oil intake ameliorates inflammation, endothelial dysfunction, thrombosis and enhances carbohydrate metabolism. The study concluded that the wide range of anti-atherogenic effects associated with olive oil consumption could explain the low rate of cardiovascular mortality found in Southern European Mediterranean countries, in comparison with other western countries, despite a high prevalence of coronary heart disease risk factors. Oleocanthal is a type of natural phenolic compound found in extra-virgin olive oil. In laboratory experiments with mice, researchers discovered that oleocanthal helps shuttle the abnormal Alzheimer's disease proteins out of the brain. Researchers have discovered that Alzheimer's disease rates are lower in Mediterranean countries, where consumption of olive oil is higher than anywhere else in the world. A study published in the journal Chemical Neuroscience set out to determine whether oleocanthal might help reduce the accumulation of beta-amyloid, believed to be the culprit of Alzheimer's disease (AD) by tracking the effects of oleocanthal in the cultured brain cells and brains of laboratory mice. It was found that in both cultured brain cells and the mice's brains themselves oleocanthal consistently boosted the production of two proteins and key enzymes known to be vital in the removal of beta-amyloid from the brain. The study authors concluded “Extra-virgin olive oil-derived oleocanthal associated with the consumption of Mediterranean diet has the potential to reduce the risk of AD or related neurodegenerative dementias. Other investigators carried out a study demonstrating that extra virgin olive oil may protect the liver from oxidative stress. Oxidative stress refers to cell damage associated with the chemical reaction between free radicals and other molecules in the body. Put simply, oxidative stress equates to cell damage. A study, published in BioMed Central, reported that laboratory rats exposed to a moderately toxic herbicide that were fed on a diet containing olive oil were partially protected from liver damage. The authors concluded that there is growing evidence that olive oil, an integral ingredient in the Mediterranean diet may have great health benefits including a reduction in coronary heart disease risk, the prevention of some cancers, the modification of immune and inflammatory responses and protection against oxidative damage of hepatic tissue.

Hemp oil: Hemp oil is an aliment obtained by pressing the raw seeds of the hemp plant, scientifically called Cannabis Sativa. Hemp seeds are rich in protein, polyunsaturated fatty acids, omega 6, omega 3 and insoluble fiber. They are a good source of tocopherols, or vitamin E antioxidants. They are packed with minerals such as potassium, magnesium, iron, zinc, calcium, phosphorus and microelements such as strontium, thorium, arsenic and chromium. High in essential fatty acids, omega 6 and omega 3, hemp oil is valuable in increasing immunity, counteracting aging skin and improving cardiovascular health. Several studies show that the linoleic acid present in hemp oil can slow down the aging process and fight psoriasis. Hemp is the only edible seed that contains gamma-linolenic acid, which is eventually converted to the protective hormone prostaglandin PGE1 that regulates hormonal balance and supports menopausal health. Due to its high content of omega 3 and omega 6 fatty acids, hemp oil has a composition similar to skin lipids, which makes it an excellent natural emollient and moisturizer. It is especially useful for dry, tired or dehydrated skin and nails. It increases skin elasticity and water retention capacity in tissues. Pure hemp oil can be used to treat dry hair and is often included in hair conditioners. Hemp oil, the only vegetable oil to contain omega-3 and omega-6 fatty acids in the optimal ratio (3:1), helps lower cholesterol levels by accelerating metabolic processes. With a faster metabolism, fats burn at a quicker rate and are not deposited on the artery walls. Due to its low carbohydrate and sugar content, hemp oil is excellent for diabetics. The nutrients present in it can help moderate blood sugar levels. Research shows that psoriasis is caused by a deficiency of omega-6 fatty acids in the body. The fatty acids present in hemp oil help improve skin oxygenation and hydration. Omega-3 and omega-6 fatty acids also improve immunity and regulate intestinal flora, thus building a natural barrier against microbes and increasing the resilience of the body. Essential fatty acids are necessary for a healthy cell membrane structure. They also prevent the demyelination, the destruction of the myelin sheath (a membrane that protects the nerve cells). Like other compounds high in omega-3s, hemp oil can thin the blood, reducing blood clots and varicose veins.

Flax seed oil: According to the Flax Council of Canada, there are numerous benefits associated with flax seed oil. ALA (alpha-linolenic acid) constitutes 57% of the total fatty acids in flax, making flax the richest source of ALA in the North American diet. Every tablespoon of flaxseed oil contains 8 grams of ALA, a polyunsaturated fatty acid. Flaxseed oil has been found to be beneficial for those who suffer from Crohn's Disease and colitis. Several studies have found that this oil seems to be able to calm the inner lining of inflamed intestines. Another benefit exists as there is a high content of mucilage in flax and this is an effective natural laxative. Flaxseed oil is beneficial in helping to reduce high cholesterol. Evidence indicates that those who eat a lot of ALA are less likely to suffer a fatal heart attack. It also reduces high blood pressure and helps those afflicted with Sjogren's syndrome. Flaxseed oil also helps to: reduce the risk of cancer, aid in the growth of healthy hair and nails, promote healthy skin, reduce menopause symptoms and plays a role in burning body fat.

Aloe Vera Juice: maintains a healthy balance between acidity and alkalinity, which is influenced by the foods we eat. The juice makes our inner system more alkaline which reduces the bad effects of acidic foodstuffs. The optimum structure of foods should be 80% alkaline and 20% acidic. This juice helps by alkalizing our system and diseases that do not survive in such an environment can be staved off. This juice absorbs toxins from the gastrointestinal tract, which travel through the alimentary canal to be discarded. The polysaccharides present in aloe juice help the production of macrophages, which defend against viruses and bacteria. The juice also contains anti-oxidants, that reduce the amount of free-radicals. It removes toxins, enhances the immune system and maintains a healthy digestion. Medical studies have shown that aloe juice can control levels of (β) sitosterol, a type of sterol with a similar chemical structure to cholesterol, in the blood. It prevents its absorption and helps to reduce overall cholesterol levels in the body, hence promoting good health. Aloe juice contains over 12 different substances that ameliorate inflammation. The juice is an adaptogen that can regulate the organism and make bowel movements more regular. It also eases acid reflux, irritable bowel syndrome and aids the body in ridding itself of worms because it is a vermifuge. There are studies that show some improvements in glucose levels when patients drink aloe vera juice because of three components thereof: lectins, mannans, and anthraquinones. This juice is a rich mixture of vitamins and minerals. It contains: Vitamins (A, C, E), contingents of B (including B1, B2, B3, B6 & B12), folic acid & choline. Moreover, it has over twenty different minerals, including calcium, sodium, potassium, selenium and iron. It also contains fatty acids and 8 essential amino acids. Aloe vera gel has many applications when used as a topical solution to treat skin conditions. It also has important secondary benefits because it works as a moisturizer and has anti-aging properties that keep skin looking young and fresh. Drinking AV juice keeps one hydrated. Additionally, it gives the body the building blocks it needs to replenish old cells and regenerate. It reduces acne and blemishes, works internally to supply vitamins and nutrients the body needs.

Turmeric: contains compounds with medicinal properties. These compounds are called curcuminoids, the most important of which is curcumin. Curcumin is the main active ingredient in turmeric. It has powerful anti-inflammatory effects and is a very strong antioxidant. Curcumin is strongly anti-inflammatory. Curcumin actually targets multiple steps in the inflammatory pathway, at the molecular level. Curcumin blocks NF-kB, a molecule that travels into the nuclei of cells and turns on genes related to inflammation. NF-kB is believed to play a major role in many chronic diseases. Curcumin is a potent antioxidant that can neutralize free radicals due to its chemical structure But curcumin also boosts the activity of the body's own antioxidant enzymes. Previously, it was believed that neurons weren't able to divide and multiply after early childhood. However, neurons are capable of forming new connections, but in certain areas of the brain, they can also multiply and increase in number. One of the main drivers of this process is Brain-Derived Neurotrophic Factor (BDNF), which is a type of growth hormone that functions in the brain. Many common brain disorders have been linked to decreased levels of this hormone. This includes depression and Alzheimer's disease. Interestingly, curcumin can increase brain levels of BDNF. Curcumin may also help reverse many steps in the heart disease process. Perhaps the main benefit of curcumin when it comes to heart disease, is improving the function of the endothelium, which is the lining of the blood vessels. It is well known that endothelial dysfunction is a major driver of heart disease and involves an inability of the endothelium to regulate blood pressure, blood clotting and various other factors. Several studies suggest that curcumin leads to improvements in endothelial function. One study shows that is as effective as exercise, another shows that it works as well as the drug Atorvastatin. Researchers have been studying curcumin as a beneficial herb in cancer treatment. It can affect cancer growth, development and spread at the molecular level. Studies have shown that it can reduce angiogenesis (growth of new blood vessels in tumors), metastasis (spread of cancer), as well as contributing to the death of cancerous cells. Multiple studies have shown that curcumin can reduce the growth of cancerous cells in the laboratory and inhibit the growth of tumors in test animals. There is some evidence that it may help prevent cancer from occurring in the first place, especially cancers of the digestive system (like colorectal cancer). Curcumin has been shown to cross the blood-brain barrier. It is known that inflammation and oxidative damage play a role in Alzheimer's disease. One key feature of Alzheimer's disease is a buildup of protein tangles called Amyloid plaques. Studies show that curcumin can help clear these plaques. Arthritis generally involves some sort of inflammation in the joints. Several studies have shown that curcumin was even more effective against arthritis than some anti-inflammatory drugs.

Ginger: Gingerol is the main bioactive compound in ginger, responsible for much of its medicinal properties. It has powerful anti-inflammatory and antioxidant effects. Ginger has been shown to be effective against exercise-induced muscle pain. In one study, consuming 2 grams of ginger per day, for 11 days, significantly reduced muscle pain in people performing elbow exercises. Ginger does not have an immediate impact, but may be effective at reducing the day-to-day progression of muscle pain. These effects are believed to be mediated by its anti-inflammatory properties. In a controlled trial of 247 people with osteoarthritis of the knee, those who took ginger extract had less pain and required less pain medication. Ginger may also have powerful anti-diabetic properties and protect against heart disease. There is evidence that ginger can enhance brain function directly and inhibit the growth of many different types of bacteria. It may also be effective against the RSV virus, a common cause of respiratory infections.

Cinnamon: is very high in cinnamaldehyde, which is responsible for most of cinnamon's powerful effects on health and metabolism. Cinnamon's ingredients, antioxidants, which have potent anti-inflammatory activity. Cinnamon can dramatically reduce insulin resistance by lowering blood sugar. Numerous human trials have confirmed the anti-diabetic effects of cinnamon, showing that it can lower fasting blood sugar levels by up to 10-29%. Two compounds found in cinnamon appear to inhibit the buildup of a protein called tau in the brain, which is one of the hallmarks of Alzheimer's disease. In a study looking at mice with Parkinson's disease, cinnamon helped to protect neurons, normalize neurotransmitter levels and improve motor function. Cinnamon oil has been shown to effectively treat respiratory tract infections caused by fungi. It can also inhibit the growth of certain bacteria, including Listeria and Salmonella. The antimicrobial effects of cinnamon may also help prevent tooth decay and reduce bad breath.

Garlic: It is now known that most of the health benefits of garlic are caused by allicin, formed when a garlic clove is chopped, crushed or chewed. A 1 ounce (28 grams) serving of garlic contains: manganese: 23% of the RDA, vitamin B6: 17% of the RDA, vitamin C: 15% of the RDA, selenium: 6% of the RDA, fiber: 1 gram and significant amounts of calcium, copper, potassium, phosphorus, iron and vitamin B1. One large 12-week study found that a daily garlic supplement reduced the number of colds by 63% compared with placebo. The average length of cold symptoms was also reduced by 70%, from 5 days in placebo to just 1.5 days in the garlic group. Another study found that a high dose of garlic extract (2.56 grams per day) can reduce the number of days sick with cold or flu by 61%. Human studies have found garlic supplementation to have a significant impact on reducing blood pressure in people with high blood pressure. Garlic can lower Total and LDL cholesterol. Garlic contains antioxidants that support the body's protective mechanisms against oxidative damage. High doses of garlic supplementation have been shown to increase antioxidant enzymes in humans, as well as significantly reduce oxidative stress in those with high blood pressure. The combined effects on reducing cholesterol and blood pressure, as well as the antioxidant properties, may help prevent common brain diseases like Alzheimer's disease and dementia.

Apple cider vinegar: contains a tiny amount of potassium and some amino acids and antioxidants. Several risk factors of cardiovascular disease have been shown to be improved by vinegar consumption; e.g., lowering cholesterol and triglyceride levels. Apple cider vinegar also contains the antioxidant chlorogenic acid, which has been shown to protect LDL cholesterol particles from becoming oxidized, a crucial step in the heart disease process. There are also some studies showing that vinegar reduces blood pressure.

Vanilla Extract: Only ten percent of the world's vanilla beans are picked when perfectly mature. This vanilla extract harvests the fully vine-ripened organic beans and is cured to assure a delicate, intense, beautiful vanilla. Through cold extraction, this exotic essence is captured in a bottle.

Orange: Fruit rich in Vitamin C

Agave Syrup: To produce agave syrup from the Agave americana and A. tequilana plants, the leaves are cut off the plant after it has aged seven to fourteen years. The juice is then extracted from the core of the agave, called the piña. The juice is filtered, then heated to break the complex components (the polysaccharides) into simple sugars. The main polysaccharide is called inulin or fructosan and is mostly fructose. This filtered juice is then concentrated to a syrupy liquid, slightly thinner than honey. Its color varies from light- to dark-amber, depending on the degree of processing.

Agave salmiana is processed differently from Agave tequiliana. As the plant develops, it starts to grow a stalk called a quiote. The stalk is cut off before it fully grows, creating a hole in the center of the plant that fills with a liquid called aguamiel. The liquid is collected daily. The liquid is then heated, breaking down its complex components into fructose and glucose and preventing it from fermenting into pulque.

An alternative method used to process the agave juice without heat is described in a United States patent for a process that uses enzymes derived from the mold Aspergillus niger to convert the inulin-rich extract into fructose. Aspergillus niger, a fungus commonly used in industrial fermentations, is “generally recognized as safe” (GRAS) by the U.S. Food and Drug Administration.

Agave nectar consists primarily of fructose and glucose. One source gives 47% fructose and 16% glucose; another gives 56% fructose and 20% glucose.

Liquid MultiVitamin:

### Multi-Vitamin Supplement With Whole Foods, Herbs, Enzymes, and

Vitamin A (as palmitate)
Vitamin C (as ascorbic acid)
Vitamin D (as ergocalciferol)
Vitamin E (as d-alpha
tocopheryl acetate)
Vitamin K (as phytonadione)
Thiamin (vitamin B1)
(as thiamine HCl)
Riboflavin (vitamin B2)
Niacin (as niacinamide)
Vitamin B6 (as pyridoxine HCl)
Folate (as folic acid)
Vitamin B12 (as cyanocobalamin)

### Biotin

### Pantothenic Acid

(as calcium pantothenate)
Calcium (as citrate complex)
Iron (as aminoate complex)
Iodine (as potassium iodide)
Magnesium (as citrate complex)
Zinc (as gluconate)
Selenium (as selenomethionine,
aminoate complex)
Copper (as aminoate
complex)
Manganese (as gluconate)
Chromium (as polynicotinate)‡
Potassium (as citrate, iodide)

### Inositol

Choline (as bitartrate)
Lutein (active carotenoid from
marigold flower extract)††

Having too much acid in the body can weaken all systems since minerals must be borrowed from bones, teeth and organs to neutralize excess acidity. High acid levels, or acidosis, might lead to diarrhea, osteoporosis, anxiety, rheumatoid arthritis and impaired immune function. Acidosis could also interfere with your cells' ability to heal or produce energy and also allow cancer to flourish. Cancer cells thrive in acidic environments. The immediate side effects of drinking alkaline water may include headaches, fatigue, muscle aches, a runny nose, and other flu-like symptoms. Also, the stool will likely be darker, softer, or more odorous and one may experience gas. These side-effects are a good sign, as they are directly related to the detoxification that the body is undergoing as the micro-clustered water penetrates deep into your tissues and expels acid waste and toxins that have accumulated over time. Side-effects will subside once the body has become accustomed to the water, anywhere from 2-3 days to 2-3 weeks, depending on each individual and the level of detoxification that the body needs to undergo.

Proper hydration sustains the body's physiological responses in all activities, but the physical and mental stresses of athletic training and competition magnify the importance of hydration. Numerous studies conducted on athletes since the 1940s have demonstrated that even mild dehydration impairs performance. Alkaline ionized water is micro-clustered, so it can be absorbed at a faster rate, helping with rehydration of the body's tissues. Furthermore, blood oxygen levels increase with regular use of alkaline water. Providing the muscles tissue and organs with high amounts of oxygen and improving stamina and endurance. Water that has been alkalized also helps to neutralize the exercise induced lactic acid build up in the muscles, and improves recovery time.

Popular belief suggested that lactate was produced in muscles due to an insufficient supply of oxygen as exercise intensity increased. These ideas also gave rise to the notion of the ‘lactic acid burn’ and that lactic acid was responsible for fatigue during high-intensity exercise. However, research has revealed that lactate is formed and used continuously, even when muscles are working with plenty of oxygen. In the human body most lactic acid is present in the form of lactate. During vigorous exercise there is an increase in the acidity in the blood and tissues. However, lactate makes a very small contribution to this. When one is exercising hard, ATP (the ‘energy currency of life’) is broken down to release energy for muscle contraction. Each time an ATP molecule is broken down, one hydrogen ion is released. Its actually the accumulation of hydrogen ions which reduce pH (a measure of acidity/alkalinity) and may be associated with ‘the burn’. So how and why is lactate produced? As exercise intensity increases, the body requires more energy more quickly and energy metabolism shifts to use more carbohydrate and less fat. Carbohydrate in the form of glucose is broken down in a chemical reaction called ‘glycolysis’. One of the end-products of this reaction is a molecule called ‘pyruvate’ which begins to accumulate along with the dreaded hydrogen ions. However, pyruvate absorbs hydrogen ions in a ‘reduction’ reaction, which forms lactate. In stark contrast to our commentator's contentions, lactate actually acts as a buffer—imagine lactate as an ‘sponge’, mopping up ions to control pH in the muscle. However, the speed of the ‘mopping up’ process has a limit. When this limit is reached, hydrogen ions accumulate, pH begins to drop and we feel the burn, but if our muscles did not produce lactate, fatigue would occur much more quickly.

The body uses lactate in a number of ways:

a) Lactate minimizes acidity in the blood and tissues As described above, lactate is the end result of pyruvate's reaction with hydrogen ions, which reduces acidity and is associated with a delay in fatigue.

b) Lactate helps to preserve other fuel stores When blood lactate concentrations are elevated, the body responds by down-regulating the use of glucose and fat. In this way, lactate could be seen as preserving precious fuel stores. Also, the body is able to generate glucose from non-carbohydrate sources through a metabolic pathway called ‘Gluconeogenesis’. Lactate is transported back to the liver, enters a chemical process called the ‘Cori cycle’ and is converted into pyruvate which is then used to generate glucose.

c) Lactate is a direct source of energy for the muscles, heart and brain. Muscles can extract lactate from the blood and oxidize it directly. This means that lactate can be used as a source of energy for muscle contraction. In some cases, the body actually prefers to use lactate as a fuel. Some research has illustrated that, during moderate intensity exercise, lactate from the working muscles may be the primary fuel source for the heart.

d) Lactate acts as a ‘shuttle’ to transfer fuel between tissues. Muscle glycogen stores are local, supplying the muscle they are stored within. This means that muscle glycogen stored in arms can't be directly transported to the legs. However, research suggests that lactate can move both within and between cells. Consequently, during heavy exercise where high rates of glucose and glycogen breakdown result in the accumulation of increasing amounts of lactate, lactate can be ‘shuttled’ from this site to other sites where is can be used as a fuel source or converted back into glucose.

e) Lactate is an important ‘signaling molecule’. Lactate may play the role of a signaling hormone, with a range of possible consequences. These include up regulating the expression of genes associated with the use of lactate as a fuel, helping the body metabolize lactate more effectively. Lactate may even stimulate an increase in formation of new mitochondria; an important adaptation in endurance performance as mitochondrion represent the ‘energy factories’ of cells. It has also been suggested that lactate can influence lipolysis (the breakdown of fat for fuel).

Lemons: According to the American Heart Association, eating higher amounts of citrus fruits may lower ischemic stroke risk for women. As an excellent source of the powerful antioxidant vitamin C, lemons and lemon juice can help fight the formation of free radicals known to cause cancer. The antioxidant vitamin C, when eaten in its natural form or applied topically, can help to fight skin damage caused by the sun and pollution, reduce wrinkles and improve overall skin texture. The risks for developing asthma are lower in people who consume a high amount of certain nutrients, one of these being vitamin C. Iron deficiency is one of the most common nutrient deficiencies in developed countries and a leading cause of anemia. Pairing foods that are high in vitamin C with foods that are iron-rich will maximize the body's ability to absorb iron. Foods that are high in vitamin C and other antioxidants can help the immune system battle germs that cause a cold or flu. Many studies have suggested that increasing consumption of plant foods like lemons decreases the risk of obesity, diabetes, heart disease and overall mortality while promoting a healthy complexion, increased energy, and overall lower weight.

Various modifications may be made without departing from the spirit and scope of the invention. For example, translucent substrates may be utilized instead of transparent substrates.

While all of the ingredients, individually, have implications of health benefits, it is the specific combination of the ingredients with high alkaline ionized pH water that achieve the unexpected results. The results achieved include extreme relief, and in some instance, complete relief from psoriasis symptoms, neuropathy, muscle weakness, reduction in elevated insulin levels in Type II diabetes, reduction in frequency, intensity, and sometimes overall occurrence of migraine headaches, relief of joint pain, and improved symptoms in COPD. The properties of the coconut oil, acting as a carrier oil across the blood brain barrier and to the liver, assist in the delivery of the other various ingredients to the areas of the body in need of assistance and relief. It is the specific combination of these ingredients that reduce the acidity of the body, as well as the degree of inflammation in the body, to allow the body to absorb and utilize the ingredients, maximizing their benefit and relief to free the immune system from oxidative stress to the point the immune system can recover. When the immune system is no longer constantly and extensively taxed, it allows it to resume natural function and strengthen to be able to better handle the stress caused by the inflammation. The enrichment of this process begins the cycle of repair and healing by the body, itself, and is continually strengthened by the body's absorption of the combined ingredients.

Additionally, the specific combination achieves decreased recovery time and increased performance in athletes. The decreased recovery time manifests in shortened time to peak performance after exertion, most notably displayed after extreme exertion. Additionally, the increased performance output of athletes has been documented, while taking the combination of ingredients twice daily.

What is claimed is:

1. A nutraceutical composition comprising water having an alkaline pH and a plurality of members of the group consisting of orange extract, vanilla extract, apple honey, agave syrup, apple cider vinegar, olive oil, coconut oil, liquid multivitamin, aloe vera juice, flax seed oil, hemp oil, and elephant garlic, wherein each of said plurality of members is present in an amount effective to ameliorate the effects of inflammation and raise energy levels when administered to a mammal.

2. The composition of claim 1 wherein said plurality of members comprises olive oil, coconut oil, liquid multivitamin, aloe vera juice, flax seed oil, hemp oil, garlic, ounces cinnamon, turmeric, ginger, and lemons.

3. The composition of claim 2 comprising said members in the proportions:
60-120 ounces alkaline water
1.5-3 ounces olive oil
1.5-3 ounces coconut oil
1.5-3 ounces liquid multivitamin
1.5-3 ounces aloe vera juice
0.5-1 ounce flax seed oil
0.5-1 ounce hemp oil,
1.5-3 ounces garlic.
1.5-3 ounces cinnamon
1.5-3 ounce turmeric
1.5 3 ounces ginger, and
3 lemons

4. The composition of claim 1 comprising all of said members.

5. The composition of claim 4 comprising said members in the proportions:
60-120 ounces water,
0.5-1 ounce orange extract,
0.5-1 ounce vanilla extract,
0.5-1 ounce elderberry syrup,
0.5-1 ounce apple honey,
1-2 ounces agave syrup,
0.5-1 ounce apple cider vinegar,
1.5-3 ounces olive oil,
1.5-3 ounces coconut oil,
1.5-3 ounces liquid multivitamin,
1.5-3 ounces aloe vera juice,
0.5-1 ounce flax seed oil,
0.5-1 ounce hemp oil,
1.5-3 ounces elephant garlic,
1.5-3 ounces cinnamon,
1.5-3 ounce turmeric,
1.5 3 ounces ginger, and
3 lemons

6. The composition of claim 1 wherein the pH of said water is between 9+ and 14+.

7. A method for the amelioration of the effects of inflammation and raising energy levels in a mammal in need thereof comprising the administration thereto of a nutraceutical composition according to claim 1 for a time sufficient to effect said amelioration.

8. A method according to claim 6 wherein said method comprises administering 8 ounces of said nutraceutical composition to said mammal once or twice a day.
